(19) United States
(12) Patent Application Publication
(10) Pub. No.: US 2011/0045515 A1
(43) Pub. Date: Feb. 24, 2011

(54) METHOD TO DETECT HEMOLYTIC STREPTOCOCCUS AND OPTOELECTRICALLY DETERMINE RESULTS

(76) Inventors: Craig J. Bell, East Swanzey, NH (US); Leroy E. Mosher, Gilsum, NH (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/866,851

(22) PCT Filed: Jan. 8, 2009
(86) PCT No.: PCT/US09/30385

§ 371 Date: Nov. 15, 2010

Related U.S. Application Data
(60) Provisional application No. 61/019,756, filed on Jan. 8, 2008.

(51) Int. Cl. C12Q 1/48; C12Q 1/44; C12Q 1/37; C12Q 1/26; C12Q 1/32; C12Q 1/28; C12Q 1/02; C12M 1/34

(52) U.S. Cl. 435/15; 435/19; 435/23; 435/25; 435/26; 435/28; 435/29; 435/288.7

(57) ABSTRACT

A reagent is provided for the detection of an exotoxin protein produced by a betahemolytic streptococcus bacteria suspected of being present in a host biological fluid collected from a subject. A kit is provided that is readily usable by an unskilled user and merely requires that an element of the kit be contacted with a biological sample and that element is then subjected to electromagnetic spectral energy. The incident electromagnetic spectral energy then reacts with the exotoxin protein indicator and can be reliably measured by an electromagnetic spectral emission. The emission is measured by a reporting module and is displayed to the user in a form recognized by the user's sensory systems; sight, sound, etc. or a combination thereof.

### CROSS-REFERENCE TO RELATED APPLICATION

This application claims priority of U.S. Provisional Patent Application Ser. No. 61/019,756 filed Jan. 8, 2008, which is incorporated herein by reference.

### FIELD OF THE INVENTION

The present invention in general relates to diagnostic testing for the presence or absence of a biomarker in a biological sample, and in particular to a rapid test for detecting clinically significant strains of Streptococcus bacteria.

### BACKGROUND OF THE INVENTION

Strep throat is an infection of the pharynx caused predominately by the bacteria Streptococcus pyogenes. The pharynx is that part of the throat between the tonsils and the larynx, or voice box. The main pathogenic beta-hemolytic strep groups for humans are A, C and G. More than 90% of streptococcal disease in humans may be caused by Group A beta-hemolytic strep (GABHS), although Group C is becoming increasingly recognized as an under-diagnosed condition.

Streptococcus pyogenes is the bacterial cause of several human infections including acute pharyngitis, impetigo, acute rheumatic fever, and scarlet fever. The particular bacterium associated with these diseases are beta-hemolytic streptococci (BHS) of Groups A, C and G, of which Group A is the most dominant pathogen.

The bacteria that cause streptococcal infection such as strep throat emit toxins that result in inflammation. The initial locale of the infection is the pharyngeal mucosa. These toxins are central in facilitating the progression of the infection. Symptoms of strep throat include a sore throat that starts suddenly, without runny nose or congestion. The throat is extremely red, and swallowing is painful. White patches typically appear on the tonsils, and lymph nodes in the neck swell. Symptoms may also include fever, headache, loss of appetite and fatigue. Children with strep throat may also exhibit nausea, vomiting and abdominal distress.

Existing tests for determining when severe sore throat symptoms may be a strep infection, such as GABHS, require a visit to a physician's office or clinical laboratory. The most commonly used in-office test is an antigen-based test, specific to GABHS. These rapid strep tests require a deep swab sample of the mucus from the pharyngeal area, which is prepared using one or two reagent chemicals. The test is considered adequate for Strep A (GABHS) positive readings (sensitivity), and takes about 3-15 minutes, but negative readings (specificity) may require additional testing. When a negative rapid strep test occurs, it is common practice to perform a laboratory cell culture to confirm or rule out the presence of a Strep A infection. The culture is required owing to a high incidence of false negatives associated with the antigen specificity of current tests. Exemplary of these tests are those disclosed in U.S. Pat. Nos. 4,863,875; 5,374,538 and 6,030,835.

People who may be at risk for serious complications from strep infection include people who have chronic conditions such as diabetes, weakened immune systems or immunodeficiency disorders. Serious complications from untreated strep infection include otitis media, peritonsillar abscesses, meningitis, peritonitis, scarlet fever and rheumatic fever. Prompt diagnosis and treatment with antibiotics is the best way to prevent infection spread and complications.

The current rapid tests require swabbing the back of the throat and tonsils to obtain a mucus sample and transferring the sample to a container or test paper. The swabbing of the throat represents a traumatic event for a patient, as well as the healthcare worker. The collection of a throat swab is made all the more difficult with pediatric patients who represent a strep-vulnerable population. With the current antigen-based tests the addition of two or more reagents is required before a visual check for the development of a color indicator. The color development is a result of GABHS antigens reacting with the antibodies introduced by the test.

The methodology is sufficiently complicated to require a laboratory technician or healthcare professional to properly perform the test and it is too complicated for use by non-professionals. Additionally, the antigen specificity of these existing tests is susceptible to false negative results for variant strains and groups of BHS. Group C BHS detection is becoming increasingly important as an epidemiological concern.

Most sore throat symptoms, however, are due to upper respiratory viruses not bacteria, and do not require immediate or extended medical care. Specifically, Group A beta-hemolytic streptococci is cultured in only approximately 15% to 20% of children with sore throats. In other words, as many as 80% of office visits are unnecessary, and could be avoided if a means were available for screening patients with sore throat symptoms before they seek advanced medical treatment, to determine if the cause of the symptoms is associated with a virus or bacteria.

BHS Groups A, C, and G produce toxins that are known as spreading agents or invasions. One such toxin that has been well documented is streptokinase. Streptokinase is specific to these several forms of streptococcal bacteria, which makes it a potentially valuable biomarker for the presence of the bacteria. Streptokinase possesses no intrinsic catalytic activity but binds to plasminogen resulting in conformational expression of an active catalytic site on the zymogen without the usual strict requirement for peptide bond cleavage. Plasminogen is the zymogen of the broad-spectrum serine protease plasmin, which degrades fibrin clots and other extracellular matrix (ECM) components such as fibronectin, laminin, vitronectin, and proteoglycans. Plasminogen is activated to its enzyme state (plasmin) by the host activator tissue plasminogen activator. Plasminogen activation is a critical component in establishing invasive bacterial infections. Subversion of the host plasminogen system renders a pathogen capable of degrading ECM proteins and activating a cascade of metalloproteases, thereby conferring the potential to invade host tissue barriers. Plasmin is subsequently produced by proteolytic cleavage and the resulting streptokinase-plasmin complex propagates plasminogen activation through expression of a substrate recognition exosite.

Direct visual detection of an enzymatic substrate cleavage by a BHS exotoxic protein are known to overcome the antigenic specificity limitations of antibody based test, as embodied in U.S. Pat. No. 7,316,910. However, direct visual detection of a color change is subjective based on visual acuity of the user, sample concentration, and substrate number.

Thus, there exists a need for a non-antigen specific rapid test for the presence of clinically significant beta-hemolytic streptococcus (Groups A, C, and G) in a bodily fluid that is operative independent of a mucosal swab and additional purification. Additionally, there exists a need for a rapid beta-hemolytic streptococcus test that could be amenable to home use as a prescreen for consultation with a health professional. The further need exists for the results of this test to be sensed through a signal processor to provide a quick result and eliminate the subjective human factor of viewing and comparing a color change indicative of BHS exotoxin in a sample.

### SUMMARY OF THE INVENTION

A reagent is provided for the detection of an exotoxin protein produced by a beta-hemolytic streptococcus bacteria suspected of being present in a host biological fluid collected from a subject includes a proteinaceous substrate for the exotoxin protein. The reagent is non-specific to antigenicity of the bacteria, in contrast to prior art beta-hemolytic streptococcus bacteria tests and instead reacts with exotoxin protein. The substrate is modified by a BHS exotoxin protein. This reaction of the exotoxin protein on the substrate has a spectroscopic characteristic. This reaction emits unique electromagnetic spectral emission. A spectroscopic indication of reaction between the substrate and exotoxin protein is measured with an optical electronic sensor and processor or a system where the electromagnetic spectral emission from the reaction is incident onto an indicating pigment or dye modifying its color indicating a positive or a negative result as secondary light emission, an auditory alarm, digital display, or combination thereof. An inventive process allows for human sensory detection and interpretation even if the emitted frequencies are outside of the human sensory detection limits. An enzyme inhibitor is optionally present to inhibit rogue protein modification of the substrate preventing a false positive result in the form of an electromagnetic spectral emission. Additionally, the electromagnetic spectral emission is read by an optoelectronic sensor that sends a signal to an electrical signal processor that interprets the signal and predicts the outcome through use of a mathematical algorithm or by a system in which the emitted electromagnetic spectral emission is incident onto an indicator pigment or dye to indicate a positive or a negative result. This allows the result to be provided to the user in a sensory format, within the detectable limits of human perception (light, sound, numeric, or alphanumeric output) and absent subjective viewer interpretation.

A kit is provided that is readily usable by an untrained user and merely requires that an element of the kit be contacted with a biological sample. That element is then placed into an optoelectronic reader that monitors the exotoxin-substrate reaction and provides a test result to the user in a sensory output format that is within the detectable limits of human perception, namely a secondary light emission, said digital display or combination thereof, indicating that the test is “positive” or “negative” for the presence of the biological marker for streptococcal bacteria. The kit includes a reagent for detecting an exotoxin protein produced by a beta-hemolytic streptococcus bacterium and an optoelectronic results reader to interpret the results as either positive or negative. The reagent contains a BHS exotoxin specific substrate and optionally a rogue enzyme inhibitor. The enzyme inhibitor suppresses rogue protein modification of the substrate to prevent a false positive result in the electromagnetic spectral emission as read by an optoelectronic sensor and interpreted by a processor or indicator pigment or dye. The substrate is optionally attached to a magnetic bead through conventional techniques such as biotinylation. While dispersed magnetic bead surface decorated with substrate for the target exotoxin favors a kinetically faster reaction under a given set of reaction conditions, concentrating the magnetic beads prior to sensing of electromagnetic spectral emissions indicative of exotoxin protein-substrate reaction increases detection sensitivity of the protein and therefore BHS.

### BRIEF DESCRIPTION OF THE DRAWING

The current invention is described in further detail in conjunction with the following referenced drawings:

FIG. 1A is a top view and FIG. 1B is a side view of an inventive test strip;

FIG. 2A is a top view, FIG. 2B is a side view, and FIG. 2C is a bottom view of another embodiment of the inventive test strip;

FIG. 3A is a schematic of the basic circuit of the optoelectronic reader used to determine the result of test strip, FIG. 3B is another embodiment of the schematic;

FIG. 4A is a schematic of the basic arrangement of an optoelectronic using indicator pigment to report results, FIG. 4B is another embodiment of this arrangement;

FIG. 5 shows a graph of color development/light intensity versus time of the test.

### DETAILED DESCRIPTION OF THE INVENTION

The present invention has utility as a procedurally simple test to detect an exotoxin protein produced by beta-hemolytic streptococcus. The exotoxin protein illustratively includes streptokinase, streptolysin O, streptolysin S, streptodornase, and cysteine proteinase. The presence of the exotoxin protein in a biological sample is indicative of the presence of beta-hemolytic streptococcal bacteria (BHS) in a host. Unlike current Group A BHS tests that rely on antigen-specific binding to an antibody or fragment thereof to confer specificity as to the group and strain of BHS, the present invention provides a simple indication of a generic or nonspecific BHS bacterial population being present, thereby decreasing the likelihood of a false negative test result that slows clinical antibiotic intervention, leading to disease spread among individuals and to other organ systems within a subject. Rheumatic heart disease is such a potential complication.

As used herein “beta-hemolytic streptococcus” is defined to include those groups of Streptococcus bacteria that are pathogenic through production of at least one extracellular exotoxic protein, streptokinase, streptolysin, streptodornase, hyaluronidase, or cysteine proteinase. These groups specifically include Strep A, C and G. It is appreciated that hyaluronidase and cysteine proteinase are also excreted by other organisms that are not necessarily pathogenic. Specifically, P. gingivalis produces arginine specific cysteine proteinase. Nonetheless, detection of these proteins in combination with BHS specific proteins adds to the certainty of the result.

The present invention provides a rapid detection kit for beta-hemolytic streptococcus bacteria through the reaction of an exotoxin protein produced by Group A, C, or G BHS with a substrate to emit unique electromagnetic spectral emission when exposed to incident light. Incident light operative to produce an electromagnetic spectral emission indicative of exotoxic protein-substrate interaction include ultraviolet, visible and infrared wavelengths, as specific wavelengths or a spectrum. The absorption spectrum of the substrate alone, or in combination with associated dyes or pigments, or as a complex with the exotoxin protein are an important factor in determining a suitable incident light wavelength. A preferred light source for incident light generation is a light emitting diode, although other light sources operative herein illustratively include a cold cathode ray tube, incandescent bulb, and a fluorescent bulb. The spectral emission can be measured with an optical electronic sensor and processor in which the electromagnetic emission is incident onto an indicator pigment or dye changing color to indicate a positive or a negative result. By communicating a color change through a secondary light emission, an auditory alarm, digital display, or combination thereof, objective results are provided that are otherwise outside the human sensory detection limits. Suitable substrates may include, but are not limited to, oligopeptide p-nitroanilides or oligopeptide amido-methylcoumarins that are cleaved by the BHS exotoxin protein directly or through activation of a secondary enzyme.

Streptokinase and cysteine proteinase are representative of the exotoxin BHS proteins effective to cleave a substrate. Additionally, it is appreciated that streptolysin that is produced by BHS is an exotoxin that binds to cell membranes containing cholesterol. Streptolysin thereafter oligomerizes to form large pores in the cell membrane that effectively lyse the membrane. As a result of streptolysin action, red blood cells represent a chromogenic substrate for streptolysin. In addition, it is appreciated that a synthetic membrane containing cholesterol is readily formed that encompasses a dye species that changes appearance with an optoelectronic sensor upon the lysis of the synthetic membrane. U.S. Pat. No. 4,544,545 teaches the formation of such a lipid bilayer.

Streptokinase acts on lysine-plasminogen to convert this substrate to an active enzyme; plasmin, streptokinase-plasmin, or streptokinase-plasminogen. The active enzyme in turn reacts with an oligopeptide p-nitroanilide to free a yellow-colored aniline dye or with the oligopeptide amido-methylcoumarin to free a fluorescent dye that is visualized when excited by UV light. Substrates for plasmin, streptokinase-plasmin, or a streptokinase-plasminogen complex include commercially available substrates S-2251 (D-Val-Leu-Lys-p-Nitroanilide Dichloride), S-2403 (pyroGlu-Phe-Lys-p-Nitroanilide Hydrochloride), S-2406 (pyroGlu-Leu-Lys-p-Nitroanilide Hydrochloride), 11040 (H-D-Ala-Leu-Lys-AMC), 11390 (H-D-Val-Leu-Lys-AMC) and combinations thereof. AMC as used herein denotes 7-amino-4-methyl-coumarin. It is appreciated that these are representative chromogenic and fluorogenic substrates for streptokinase and that other substrates such as chemiluminescent, and other fluorogenic and chromogenic oligopeptide substrates are operative in place of, or in combination with, the aforementioned oligopeptides. Streptokinase activity has previously been measured chromogenically. W. Tewodros et al., Microbiology Pathology 18 (1995): 53-65.

BHS cysteine proteinase is also noted to be specific towards the chromogenic oligopeptide substrate N-succinyl Phe-Ala-p-Nitroanilide and Leu-p-Nitroanilide. It is appreciated that substrates for both streptokinase and cysteine proteinase are readily included within the inventive test kit in which greater sensitivity to the presence of BHS is desired.

An additional substrate operative for the detection of BHS is a membrane having cholesterol within the membrane and containing within the membrane volume a chromophore that changes color upon membrane lysis through oligomerization of streptolysin O or S. Membranes including cholesterol that are suitable as substrates for detection of BHS streptolysin include red blood cells and lipid bilayers including cholesterol and chromophores. The chromophores typically include hemoglobin and the aforementioned nitroanilide oligopeptides. It is appreciated that as with streptokinase substrates, cysteine proteinase and streptolysin substrates are readily provided that include a chemiluminescent, fluorogenic or other chromogenic species therein. Such chemiluminescent and fluorogenic species couplable to oligopeptides are insertable into liposomal membranes are well known to the art and are described in U.S. Pat. No. 4,544,545. Streptolysin S activity alone or in combination with streptolysin O activity has also previously been measured chromogenically. A. Heath et al., Infectious Immunity 67 (1999): 5298-5305.

Preferably, a substrate for detecting an exotoxin protein produced by beta-hemolytic streptococcus is provided within or on an inert solid matrix. Suitable materials for the formation of an inert solid matrix include cellulosic materials such as filter paper, natural fibers such as cotton, linen, silk, and wool; nitrocelluloses, carboxyalkyl celluloses, synthetic polymer fabrics such as polyamides, polylactic acids, polyacrylics and sintered polyalkylene beads. If the substrate includes a fluorescent molecule, the solid matrix should have low or no fluorescing properties.

Alternatively, solution-based substrates for BHS extracellular proteins are provided in conventional buffer solutions such as PBS (phosphate buffered saline). The substrate is optionally attached to a magnetic bead through conventional techniques such as biotinylation. While dispersed magnetic bead surface decorated with substrate for the target exotoxin favors a kinetically faster reaction under a given set of reaction conditions, concentrating the magnetic beads prior to sensing of electromagnetic spectral emissions indicative of exotoxin protein-substrate reaction increases detection sensitivity of the protein and therefore BHS. The beads afford attractive attributes of both solid matrix and solution based substrates. Preferably, a buffer solution includes an antimicrobial agent to preclude substrate degradation by opportunistic micro-organisms. It is further appreciated that the shelf life of an inventive reagent and therefore a kit for performing an inventive nonspecific BHS strep test is increased by storing the reagent under cool conditions such as those found in a consumer refrigerator/freezer. In instances where substrates are in solution form, or red blood cells are provided as a substrate for streptolysin, preferably a cryopreservative is present. Typical of cryopreservative solutions are those that include 2% heta starch, 4% albumin and 7.5% dimethylsulfoxide.

Biological fluids from a host suitable for detection of BHS therein include sweat, mucosa, saliva, blood, tears, and pus. In a circumstance where one is attempting to detect BHS associated with a sore throat, the preferred biological fluid is saliva, in contrast to prior art antigenic binding that has required throat mucosa. Saliva represents a less invasive source of biological fluid for the determination as to the presence or absence of an active strep infection and is collected by buccal swab or expectoration in contrast to a throat swab. While saliva is readily collected in a home setting, a throat swab necessitates a degree of medical skill The present invention is based upon the recognition that saliva of an individual having a BHS-induced pharyngitis contains streptokinase, streptolysin, cystein proteinases and other exotoxins associated with BHS.

It should be appreciated that the various biological fluids that have been indicated as host suitable for testing for the presence or absence of BHS by detecting an exotoxin protein, such as streptokinase, also contain a vast number of other proteins. When a given substrate is found to be cleavable by rogue (non-specific and non-targeted) proteins such as; trypsin, kallikrein, tissue plasminogen activator (tPA), calpain, cystatin, kinases, peroxidases, dehydrogenases, phosphorylases, transferases, reductases, mutases, and/or isomerases; other than the particular exotoxin proteins mentioned above, a proper enzyme inhibitor(s) preferentially inhibiting the rogue proteins is used. For example, trypsin is a serine protease and a digestive enzyme produced in the pancreas and found mainly in the intestines, but also at low concentrations in the stomach and in saliva. In a preferred embodiment of saliva sampling for BHS exotoxins, trypsin enzymatic activity is suppressed to enhance detection of the BHS specific exotoxin streptokinase.

Non-BHS enzyme inhibitors are provided in a biological sample or in an inventive reagent to prevent false positive testing results by minimizing or preventing the action of the rogue proteins(s) from cleaving the substrate allowing the targeted exotoxin protein to be the only one reacting with the substrate and enhancing the sensitivity of the testing results. Inhibitors illustratively include ecotin specifically inhibiting trypsin; Pefabloc SC (Roche) broadly inhibiting a broad spectrum of serine proteases, including trypsin; formaldehyde and phenyl isocyanate which provide ribonuclease inhibition; and cystatins isolated from tick saliva which are cysteine protease inhibitors. The appropriate quantity of non-BHS enzyme inhibitor is readily determined using standard solutions with known quantities of trypsin and a uniform quantity of a target BHS exotoxin.

It is appreciated that adequate time is provided for a biological fluid sample to be pretreated with an enzyme inhibitor to suppress a false positive color change of the testing results associated with a given rogue protein. This pretreatment is preferably required when a biological fluid sample, such as human saliva, is complex in nature. By way of an example, a particular enzyme targeted by an enzyme inhibitor in the present invention is trypsin.

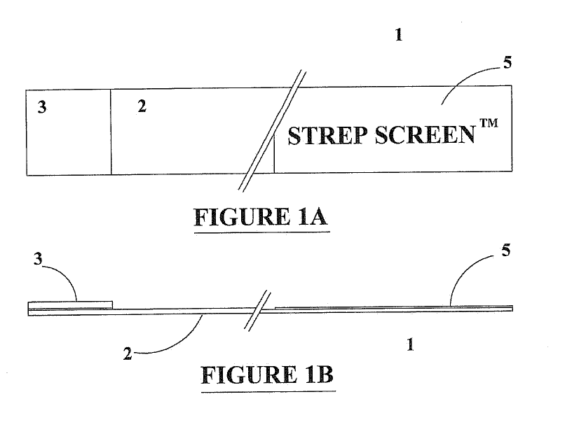

Referring now to the figures, exemplary embodiments of the present invention are provided. Referring now to the embodiment of the invention shown in FIGS. 1A and 1B, a test strip also commonly referred to as a dipstick is shown generally at 1. The test strip 1 is preferably constructed of a thermoplastic illustratively including polystyrene and polypropylene. Thermoplastic strip 2 has an exemplary size of approximately 0.25″ wide by 3″ long by 0.015″ thick. The test strip 1 has a solid matrix 3 which contains BHS reagent formula 4. Solid matrix 3 is attached to plastic strip 2 by pressure-sensitive adhesive or other common laminating means, such as heat sealing. The solid matrix is the surface of the plastic dipstick or a filter material such as Whatman Inc. papers CF 4 or BA 83. Test strip 1 has an area 5 that is used for labeling, as in a pressure-sensitive label or pad printing ink.

The reagent formula 4 is typically dispensed onto solid matrix 3 by a manual pipette, automated pipette, or other precision dispensing means currently known in the art. The substrates are optionally impregnated throughout the thickness of the matrix. Such saturation methods, including dip baths, enhances the extent of reaction with an active enzyme associated with BHS and illustratively includes streptokinase-plasminogen complex, streptokinase-plasmin, and plasmin. Alternate substrate application methods include various printing techniques are known for application of liquid reagents to carriers, e.g. micro-syringes, pens using metered pumps, direct printing and ink-jet printing. The volume of reagent formula 4 disposed is between 0.5 and 100 microliters. The indicating formula is then dried at room temperature or at an elevated temperature, but one not so elevated as to denature the formula. The drying process is assisted by vacuum and/or a desiccating agent. Reagent formula 4 is reactive with a BHS extracellular protein exotoxin directly or indirectly as a result of a complex or activation of an enzyme. Preferably, the BHS protein includes at least one of streptokinase, streptolysin O, streptolysin S, and cysteine proteinase. Preferably, the reagent fluorogenically detects streptokinase. A reagent formula for streptokinase includes at least a fluorogenic substrate H-D-Val-Leu-Lys-AMC (Peptides International) and optionally the single chain glycoprotein plasminogen (Sigma), which is the inactive precursor to the active enzyme plasmin and optionally at least one rogue enzyme inhibitor (Roche).

The optional addition of plasminogen to reagent formula enhances the reaction between the streptokinase and endogenous plasminogen with the substrate. The plasminogen is isolated from a variety of sources. Human plasminogen is obtained from pooled plasma, glu-plasminogen, lys-plasminogen, recombinant, and/or fractions of plasminogen. Highly purified lys-plasminogen is the preferred form of the zymogen for reagent formula 4 because it is 20 times more reactive than the glu-plasminogen form. Since the vast majority of plasminogen in human blood is glu-plasminogen, lys-plasminogen is manufactured from purified glu-plasminogen. By plasmin hydrolysis of the Lys76-Lys-77 peptide bond of glu-plasminogen, lys-plasminogen is formed. The process then involves a plasmin quenching process and lys-plasminogen purification process.

Biological protein stabilizers are optionally included into reagent chemistry formulation 4. Bovine serum albumin (Sigma) and Prionex (Centerchem) are protein stabilizers that improve a proteinaceous substrate shelf life.

Reaction enhancement additives are another component that can optionally be included into reagent formula 4. These additives induce a conformational change to the molecular structure of the streptokinase, the lys-plasminogen, or both to states that favor the reaction and accelerate the outcome. These additives include, but are not limited to, non-ionic detergents such as Triton (Fisher Scientific) and mammalian protein fibrin, or protein fibrinogen (Sigma) or polypeptides with a lysine binding site (poly-D-lysine).

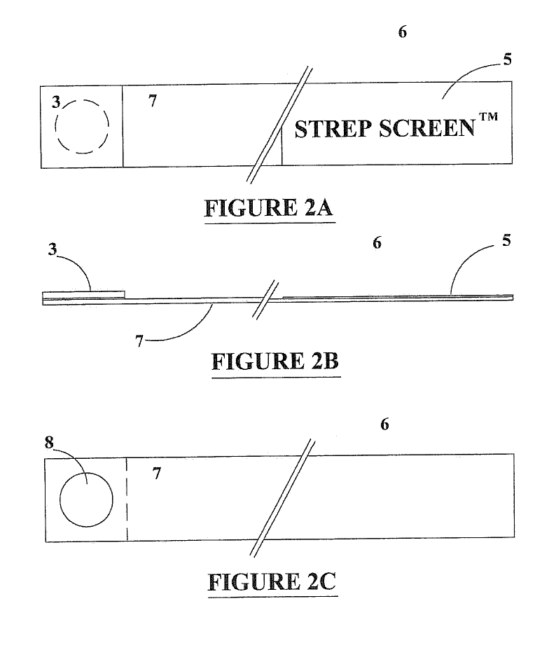

Referring to FIGS. 2A, 2B, and 2C; the test strip design 6 is the same as test strip 1 shown in FIGS. 1A and 1B with the modification of through hole 8 in thermoplastic strip 7. Through hole 8 allows an excitation frequency of electromagnetic energy to be shown to the underside of solid matrix 3. The electromagnetic energy change is monitored by an optoelectronic sensor on the opposite side of solid matrix 3 or by a system in which the emitted electromagnetic frequency(ies) is incident onto an indicator pigment changing its color, indicating a positive or a negative result.

When reagent formula 4 includes a fluorogenic substrate it is important and not immediately obvious that solid matrix 3 has low or no fluorescing properties. It is common in the paper industry to add UV brighteners that are excited by the ambient UV wavelengths and result in a whiter, brighter paper product. That is not desirable in this application as it represents background fluorescence, producing visible interference with the test result.

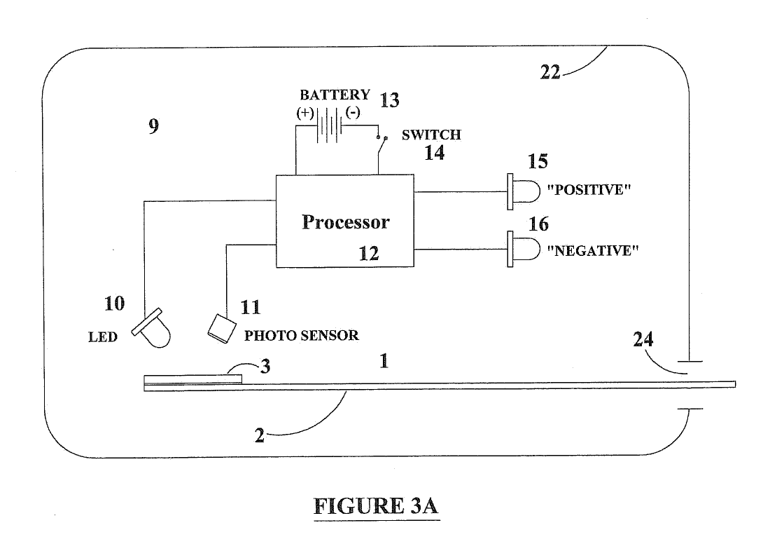

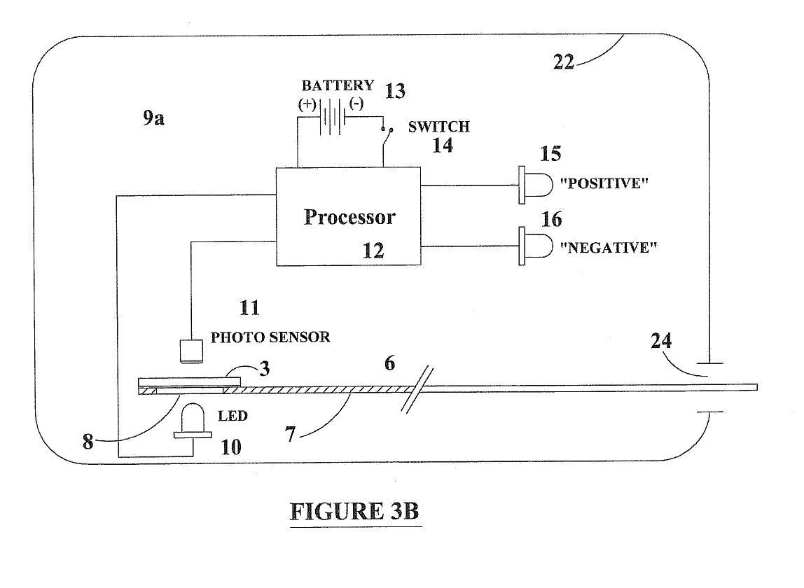

FIG. 3A depicts a test strip 1 in a test instrument represented generally at 9 including an optoelectronic reader used to determine the result of test strip 1. A housing 22 is preferably provided having an opening 24 provided through which the test strip 1 or 6 is inserted and test results are apparent by a sensory output format that is within the detectable limits of human perception (light, sound, numeric, or alphanumeric), or combination thereof. Preferably, the housing 22 is handheld and well suited for mobile test strip reading as might occur in a home, temporary clinic, or school setting through resort to a battery power source. The schematic shows LED 10 and photosensor 11 positioned to expose solid matrix 3 of test strip 1 to tuned frequency or frequencies of electromagnetic energy and to monitor solid matrix 3 for the emitted electromagnetic profile from the same side of solid matrix 3. LED 10 can provide visible white light, ultraviolet (UV) light, or other light wavelengths depending on the reagent formula 4 and the substrate requirements. Photosensor 11 can be a photodiode or a phototransistor or other form of color/light/fluorescent/electromagnetic spectral intensity measuring device. It is appreciated that hyperspectral sensing of emission from the interaction between the target exotoxin protein and the substrate can provide superior signal to noise data of a result than a single wavelength detection. Photosensors with multiple wavelength response and suitable signal processing algorithms allow for hyperspectral detection of BHS. The signal from the photosensor is sent to electrical signal processor 12, where the signal is conditioned, converted, amplified and/or interpreted through a mathematical algorithm and threshold limit comparison program(s). An illustrative example of electrical signal processor 12 is a programmable microprocessor. The results of electrical signal processor 12 can be displayed in several different means know in the art. One method is shown in schematic 9, when the result is determined to be positive for the presence of streptococcus bacteria, LED 15 lights to illuminate the word “POSITIVE” through a transparent window in the instrument's housing and when the result is determined to be negative for the presence of streptococcus bacteria, LED 16 lights to illuminate the word ‘NEGATIVE” through a transparent window in the instrument's housing. Other methods would have the words “POSITIVE” or “NEGATIVE” on a digital screen and/or have the result given from an audio chip speaking the words of “POSITIVE” or “NEGATIVE”. Also shown in schematic 9 is battery 13 as the power supply and switch 14 as the on/off control. It is appreciated that the instrument could be configured to be powered by either AC and/or DC current. What are not shown, but are optionally included, are a timer which would inform the user on incubation time for the sample to be in contact with the inhibitor(s) in the collection cup before exposing the test strip to the sample and an optional temperature controlling unit to keep the sample exposed test strip at a constant temperature and optionally at a temperature to maximize the enzymatic reaction, for example 37-40° C. without degrading the sample, the inhibitor(s), and/or reagent 4.

FIG. 3B shows alternate basic circuit schematic 9a which has test strip 6 positioned in such a way that LED 10 is exposing solid matrix 3 to electromagnetic energy through hole 8 in thermoplastic strip 7 and photosensor 11 monitoring electromagnetic frequency changes from the opposite face of solid matrix 3, where like numerals correspond to those used with respect to schematic 9 of FIG. 3A.

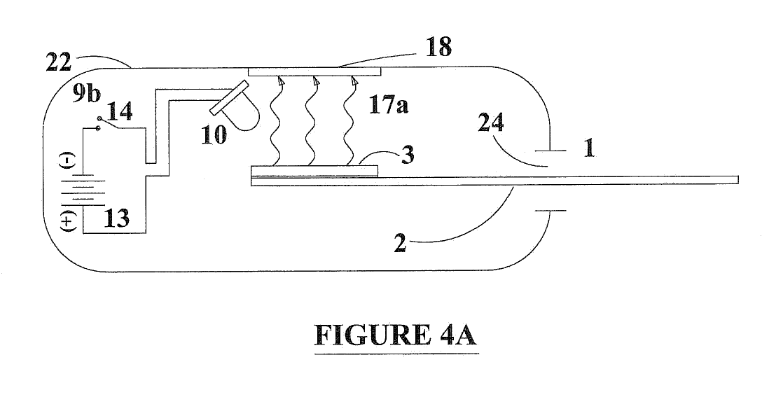

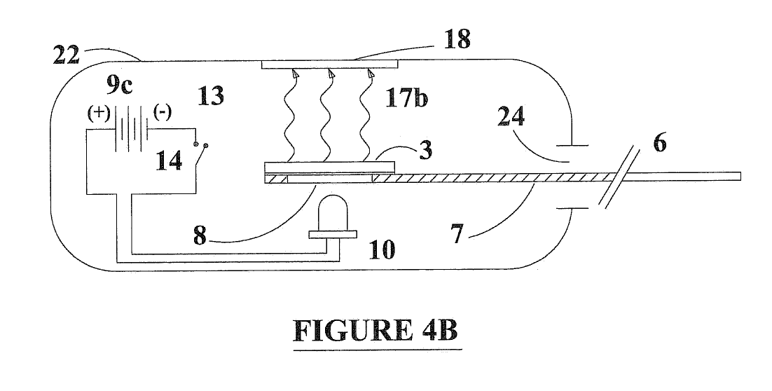

FIG. 4A shows a basic circuit schematic 9b which has test strip 1 positioned so LED 10 is exposing solid matrix 3 to a tuned frequency or frequencies of incident light, as the association between exotoxin-substrate takes place, solid matrix 3 will emit unique electromagnetic spectral emission profile 17a. Emitted spectral profile 17a is incident onto indicator pigment or dye 18 changing the color indicating a positive or a negative result. This allows for human sensory detection even if the emitted frequencies are outside of the human sensory detection limits. FIG. 4B shows schematic variation 9c, with LED 10 is positioned to expose solid matrix 3 to electromagnetic energy through hole 8 in test strip 6. Emitted frequency profile 17b is incident onto indicator pigment 18 changing its color indicating a positive or a negative result.

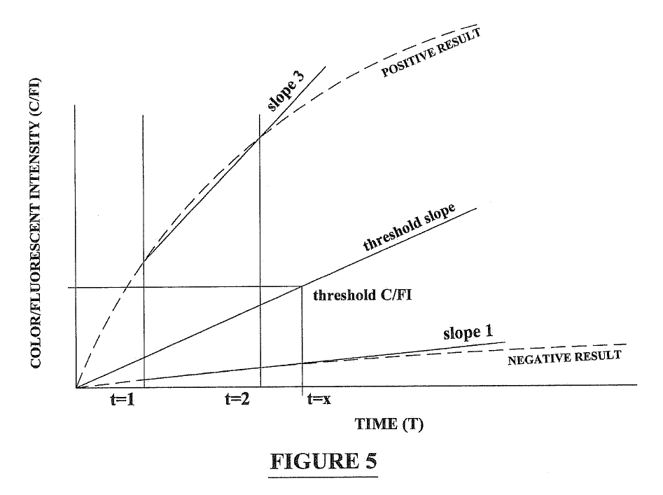

FIG. 5 is a graphic representation of the conditioned signal output of the photosensor as color/fluorescent intensity (C/FI) versus elapsed time. The graph shows a positive result, a negative result, threshold slope, and threshold C/FI level. The processor program compares the test strip C/FI at a predetermined time (t=x) to a programmed threshold C/FI level and determines in the test strip is a positive or negative result. An optional program method compares the rate increase of C/FI (slope) over a predetermined time segment (t2−t1) to that of a predetermined threshold slope. C/FI increases greater than or equal to the threshold slope are reported as a positive result and C/FI increases less than the threshold slope are reported as a negative result. These methods to determine the positive or negative result of the test strip are not to be considered the only means or methods to utilize the output from the optoelectronic sensor and that others exist in the art.

When the device is used, the patient is asked to cough and then expectorate into collection cup 6. If the embodiment has the rogue enzyme inhibitor desiccated in the cup, then the sample is incubated at room temperature for between 1 and 30 minutes. This allows time for the interfering enzymes to be inactivated before the sample is brought into contact with solid matrix 3 of test strip 1. The test instrument optionally is equipped with a timing mechanism to notify the user when the sample incubation is done. Test strip 1 is removed from a protective packaging and solid matrix 3 end is submerged in the sample for 1-2 seconds. Exposed test strip 1 is then optionally placed in a small resealable polymer bag and sealed. This bag prevents the solid matrix with sample and reagent formula 4 from drying out or otherwise changing the reaction environment, as well as containing the biologic sample.

Test strip 1 is now placed in the test instrument represented generally in FIG. 3A at 9. The testing is initiated by test instrument switch 14 by manual activation, proximity switches, latch switches, or other activation means known in the art. LED 10 illuminates sample exposed solid matrix 3 and photosensor 11 monitors the surface of solid matrix 3 for color/fluorescent intensity development versus lapse time, preferably in seconds and minutes. The biochemical reaction on solid matrix 3 requires a time of approximately between 5 and 45 minutes to develop a discernable color change at room temperature. Optionally, test strip 1 in the resealable bag is exposed to temperatures greater than room temperature, but below temperatures that could denature the proteins of reagent formula 4 and of the biological sample on solid matrix 3. Since the reaction is enzymatic, the activity increases with increasing temperature to about 40° C. The temperature increase can be achieved in the test instrument by a resistance heating element or other means known in the art. The output electrical signal from photosensor 11 is sent to electrical signal processor 12 for signal conditioning and interpretation through one of, but not limited to, the previously discussed programs. If the program determines that the color/fluorescent intensity development meets the predetermined criteria for a positive result, the test instrument reports that to the user by any of several ways including a LED backlit indicator showing “POSITIVE”, a digital screen, or an audio indicator. If the result meets the predetermined criteria for a negative result, similar means would be used to report the “NEGATIVE” result to the user.

A test strip 1 or 6 is exposed to the saliva sample and placed in test instrument shown generally at 9a in FIG. 3B. Test strip 6 has a through hole 8 in thermoplastic strip 7 which exposes the back surface of solid matrix 3. Test instrument schematic 9a shows LED 10 positioned so that when the test cycle starts it illuminates the back of solid matrix 3. Solid matrix 3 structure is such that the illumination of it and the color/fluorescent development can be monitored by photosensor 11 on the opposite surface of solid matrix 3 as shown. The use of the electrical output signal generated by photosensor 11 is processing by processor 12, and the reporting of the results are similar to that described in the previous paragraph.

Optionally, test strip 1 or 6 is exposed to the saliva sample and placed into test instrument shown generally as 9b in FIG. 4A or 9c in FIG. 4B. Solid matrix 3 of the test 1 or 6 is exposed to a tuned frequency or frequencies of electromagnetic energy. There will be a resulting biochemical reaction emission electromagnetic frequency 17a or 17b that is unique. These emission frequencies can be shifted by indicator pigment or dye 18 to provide a method for human sensory detection, even if the emitted frequencies are outside the human detection limits through generation of an electrical signal that is communicated to a user as secondary light emission, an auditory alarm, digital display, or combination thereof. The resulting outputs indicate if the test result was positive or negative in one of several sensory formats (light, sound, numeric, or alphanumeric).

It is appreciated that inventive test kits for detecting BHS in biological fluids other than saliva optionally vary in host sample aliquot volumes and reagent quantity to attain desired levels of sensitivity and specificity. Factors to achieve these variations include the design of the solid matrix, type of material, and stick design, and sample collection cup design. Preferably a solid matrix collects enough biological fluid to hydrate the indicating formula. It is appreciated that excessive liquid dilutes the reagent formula and results in a less intense fluorogenic or chromogenic reaction. Modified solid matrix designs that are employed to minimize reagent dilution are polymeric film covering of the solid matrix that allows the liquid sample to wick in at least one open edge of the matrix or through the cover's porous structure. Another solid matrix design that is optionally employed is to treat the solid matrix so the molecules of reagent formula are slowed or prevented from diffusing out of the matrix.

It is appreciated that a reagent formula includes in a single volume proteinaceous substrates for streptokinase, cysteine proteinase each alone, or in combination with a cholesterol-containing membrane reactive towards streptolysin. Alternatively, the use of two or more separate reagent formulas each specific for a different BHS exotoxin affords greater selectivity to BHS since the possibility of contamination of a biological fluid sample with two or more of the exotoxins produced by BHS or a false positive becomes much less likely. It is appreciated that the multiple separate reagent formulas are readily contained on two or more solid matrix pads on test strip 1 or test strip 6, each specific to a different BHS exotoxin. This would include a test instrument that has multiple LED illuminating lights and multiple photosensors. The signal outputs would still be fed to an electrical signal processor that is equipped to condition the signals and programmed to determine the results of multiple color/fluorescent intensity developments on multiple solid matrix pads. The results would be reported by means previously discussed.

Additionally, while in a preferred embodiment streptokinase is detected through interaction with plasminogen introduced into a reagent formula, it is appreciated that a simplified streptokinase reagent formula is operative that relies on the presence of plasminogen naturally found in the biological fluid and in such an instance, the inventive reagent formula need only include a fluorogenic oligopeptide or a p-nitroanilide containing substrate that yields a color change discernable to an unaided human eye that is a substrate for the streptokinase-plasminogen complex, streptokinase-plasmin complex or plasmin. It is appreciated that an inventive reagent formula is readily made of various concentrations of fluorogenic substrate or cholesterol containing membrane containing a fluorophor to yield different formula sensitivities, color development intensities, and color development times. A starting point for the concentrations is to make a fluorogenic substrate concentration of 1 millimolar solution and in the case of streptokinase detection, a plasminogen concentration of 300 micrograms per milliliter (μg/ml). 10-20 microliters of each solution alone, or in combination with a like amount of plasminogen solution, is placed into container 1 and let dry at room temperature for streptokinase detection.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process for detecting an exotoxin protein produced by a beta-hemolytic streptococcus bacterium being present in a biological fluid collected from a subject, comprising:
contacting said biological fluid sample with a substrate modified by the exotoxin protein of streptokinase, streptolysin O, streptolysin S, streptodornase, or cysteine proteinase;
exposing said substrate to light from a light source;
sensing an electromagnetic spectral emission from said substrate in reflective or transmissive mode with a photosensor sensitive to said electromagnetic spectral emission indicating presence of the exotoxin protein, and said electromagnetic spectral emission being due to said substrate being modified by said exotoxin protein to yield an electrical signal indicative of said electromagnetic spectral emission; and
communicating the electrical signal through an electrical signal processor to a user as secondary light emission, an auditory alarm, digital display, or combination thereof.

2. The process of claim 1 further comprising mixing said biological fluid sample from the subject with an enzyme inhibitor to form a treated sample, wherein said enzyme inhibitor inhibits rogue non-targeted enzymes in preference to the first exotoxin to prevent a false positive result absent the first exotoxin protein.

3. The process of claim 2 wherein said rogue protein is selected from the group consisting of: trypsin, kallikrein, tissue plasminogen activator (tPA), calpain, cystatin, kinases, peroxidases, dehydrogenases, phosphorylases, transferases, reductases, mutases, and/or isomerases; and the biological fluid is saliva from the subject.

4. The process of claim 1 wherein said contacting step occurs for at least a portion of the total contact time with said substrate at a temperature of between 37 and 40 degrees Celsius.

5. The process of claim 1 wherein said communicating step includes illumination of a light secondary source indicative of said electromagnetic spectral emission.

6. The process of claim 1 further comprising placing an inert solid matrix in contact with said substrate prior to said contacting step.

7. The process of claim 1 wherein said sensing step is hyperspectral.

8. The process of claim 1 wherein said electromagnetic spectral emission is sensed in transmission and a support for said substrate is transparent to the light from a light emitting diode serving as said light source.

9. The process of claim 1 wherein said electrical signal processor uses a time dependent change in said electrical signal to determine if said electromagnetic spectral emission has occurred.

10. The process of claim 1 wherein said substrate is fluorogenic or chromogenic.

11. The process of claim 1 wherein said substrate is fluorogenic or chromogenic and adhered to magnetic beads and further comprising concentrating said beads prior to said sensing step.

12. The process of claim 1 wherein the exotoxin protein is streptokinase.

13. A kit for detecting a first exotoxin associated with beta-hemolytic streptococcus bacterium in a biological sample collected from a subject comprising:
a reagent for detecting a first exotoxin protein produced by a beta-hemolytic streptococcus bacterium suspected of being present in a biological fluid collected from a subject, comprising:
a first substrate modified with a degree of specificity by the first exotoxin protein to induce an electromagnetic spectral emission;
a test strip on which said reagent is deployed;
an optical test structure for optically detecting said electromagnetic spectral emission associated and communicating to a user said electromagnetic spectral emission indicative of the first exotoxin being present through secondary light emission, auditory alarm, digital display, or combination thereof; and
instructions for the use thereof for detecting the first exotoxin associated with the presence of the beta-hemolytic streptococcus in the biological sample.

14. The kit of claim 13 wherein said reagent further comprises an enzyme inhibitor inhibiting rogue protein present in the biological fluid and not correlating with the beta-hemolytic streptococcus bacterium from modifying said substrate to prevent a false positive result of said electromagnetic spectral emission.

15. The kit of claim 13 wherein said optical test structure further comprises a light emitting diode emitting at least one light wavelength onto said substrate and a photosensor sensitive to said electromagnetic spectral emission sensing an optical emission from said substrate in reflective or transmission mode relative to said at least one light wavelength.

16. The kit of claim 15 wherein said at least one light wavelength is ultraviolet and said substrate is fluorogenic.

17. The kit of claim 15 wherein said optical test structure for optically detecting said electromagnetic spectral emission communication to the user is the secondary light emission.

18. The kit of claim 13 wherein said optical structure is battery powered.

19. The kit of claim 13 wherein said reagent further comprises magnetic beads to which said first substrate is adhered.
